(19) United States
(12) Patent Application Publication
(10) Pub. No.: US 2019/0142880 A1
(43) Pub. Date: May 16, 2019

(54) THE USE OF PHYCOCYANINS, PURIFIED OR AS PRESENT IN CYANOBACTERIAL MICROALGAE OR EXTRACTS THEREOF, AS PREBIOTICS, TO ENHANCE THE VIABILITY, GASTROINTESTINAL SURVIVAL, PATHOGEN-FIGHTING ABILITY, AND THE OVERALL HEALTH-ENHANCING PROPERTIES OF PROBIOTIC CULTURES AND PRODUCTS.

(71) Applicant: Stefano SCOGLIO, San Marino (IT)

(72) Inventors: Stefano SCOGLIO, San Marino (IT); Wally BAFFONE, Urbino (IT); Raffaella CAMPANA, Urbino (IT); Evelin COLOMBO, Urbino (IT)

(21) Appl. No.: 16/098,618

(22) PCT Filed: May 1, 2017
(86) PCT No.: PCT/EP2017/060309

§ 371 Date: Nov. 2, 2018

Related U.S. Application Data
(60) Provisional application No. 62/330,450, filed on May 2, 2016.

(51) Int. Cl. A61K 35/748; A23L 33/195; A61K 35/741; A23L 33/135; A61P 31/04; A61K 38/16; A61K 9/00

(52) U.S. Cl. CPC A61K 35/748; A23L 33/195; A61K 35/741; A23L 33/135; A61P 31/04; A23V 2250/202; A61K 9/0014; A61K 9/0034; A23V 2002/00; A23V 2200/3204; A61K 38/164

(57) ABSTRACT

The use of probiotics has become very widespread as a way to sustain gastrointestinal and general health. However, not all probiotic bacteria are effective, due to poor viability, inability to survive the gastrointestinal passage, to kill pathogens and colonize the gut. That is why pre-biotic substances are usually added to sustain, nourish, protect probiotic bacteria, make them stronger and increase their overall health-enhancing properties. We have found, for the first time, that phycocyanins from cyanobacterial algae, added to the culturing of probiotic or to probiotic products, are very effective as prebiotics, being able to strengthen probiotic bacteria's viability, resistance, pathogens-fighting ability and overall ability of probiotics to sustain human and animal health.

### BACKGROUND

The World Health Organization defines probiotics as “live micro-organisms which, when administered in adequate amounts, confer a health benefit on the host.”; and the FAO/WHO joint committee that was established afterwards used such a definition to set up specific guidelines for the evaluation of probiotics in food (1). There have been many discussions on what are the essential requisites for good and effective probiotics, but generally they can be summarized as follows:
The probiotics have to be alive when administered, and so a crucial requisite is their endurance and survival ability, as much as possible, during production, that is during the culturing process;
The next requisite is the ability of the probiotic cells to endure transportation and storage time. Both transportation and storage represent stressful times for the micro-organisms, which could be seriously compromised and weakened by these phases; clearly, the stronger the probiotic cells, the more viable they are;
Even when the first two requisites are satisfied, the next one is absolutely essential: you can manage to ingest still living probiotic cultures, but unless its probiotic cells are able to survive the passage through the human gastro-intestinal tract, those probiotics will be useless, because destroyed by the stomach acids or by the bile.
Finally, even assuming that the probiotic culture, in liquid or powder form, reaches the gut, the next essential requisite is its ability to attach to the intestinal wall and properly colonize the small intestine and or colon, effectively competing with pathogenic organisms, such as Escherichia coli or Candida albicans.

Various means to increase growth and viability, as well as acids/bile resistance and anti-microbial activity, have been sought. Probiotics mostly belong to the Lactobacillus and Bifidobacterium genera, which are health promoting bacteria of the balanced intestinal microbiota (2,3) and are able to inhibit the growth of numerous pathogens by the production of organic acids, bacteriocins, hydrogen peroxide (4). Beyond the assessment of probiotic and the development of methods to identify new probiotic microorganisms, the concept of prebiosis has become important. Prebiotics were defined as non-digestible food ingredients that beneficially influence the host by selectively stimulating the growth and/or the activity of one or a limited number of bacteria in the colon (5). The main prebiotics are non-digestible food carbohydrates, such as fibers, oligosaccharides including fructo-oligosaccharides (FOS) and galacto-oligosaccharides (GOS), resistant starch, as well as proteins or peptides originated by human digestion and utilized by microorganisms as a source of energy (6). Moreover, there is evidence that prebiotics can help to modulate the growth of gut microbiota and stimulate bacteriocin production by probiotic strains, such as Lactic Acid Bacteria (LAB) (7,8, 9). Prebiotics can influence the metabolic activity of probiotics; their combination, called “symbiotic”, favors the gut probiotic colonization and improve the quality of human life (10). In fact, symbiotics have been shown to be more effective than probiotics or prebiotics alone in patients with ulcerative colitis (11), in colorectal cancer prevention (12) or the general positive regulation of the microbiota (13).

During the development of new symbiotic products it is very important to assess prebiotic and probiotic interactions, and the influence of prebiotic on probiotic growth and antibacterial activity. Recent studies focused on symbiotics constituted by the association of prebiotic strains with oligosaccharides (14, 15) or other natural compounds (16). New approaches to symbiotic combinations with the intent to obtain synergistic effects could consider the substances contained in blue-green algae, rich in carotenoids, chlorophyll, phycocyanin and many other bioactive components.

Indeed, the core of this invention is the use of phycocyanins, especially from, but not limited to, the specific strain of microalgae Aphanizomenon flos aquae strain. We will show that in fact phycocyanins alone, or in combination with the whole cyanobacteria Aphanizomenon flos aquae, performs better than any other whole microalgae in providing the needed support to the growth and activity of lactobacilli.

Studies have been performed on the use of Spirulina or Chlorella as an addition to the probiotic culture. (17). They grew S. thermopiles TH4, L. lactic C2, and L. delbrueckii YL1 with and without the addition of 3 mg of dry S. platensis/mL biomass. After 4 h, the LAB growth promotion by S. platensis, at pH 6.8, was 13.42% for C2, 9.29% for YL1, and 8.22% for TH4, compared with the controls. After 8 h, the increase was 3.46%, 9.73%, and 7.76% for C2, YL1, and TH4, respectively, and that is probably due to a decrease in the amount of the stimulatory factors. The 3 strains treated with Spirulina reached the stationary phase at 10 h and the counts remained the same up to 20 h, while the same strains without Spirulina addition grew more slowly and continued to grow up to 20 h, reaching the same value as the supplemented ones, which means that the addition of Spirulina into the growth medium did not really significantly affect the growth and viability of the probiotic strains.

Molnár et al. (18, 19) studied the effects of Spirulina biomass on single strains of mesophilic lactic acid bacteria. Used at the rate of 3 g/dm3, Spirulina significantly increased (P<0.05) the acid production by various strains of mesophilic lactic acid bacteria. During the first 2 weeks of refrigerated storage at 4±2° C., the Spirulina biomass significantly increased (P<0.05) viability of mesophilic starter bacteria in the product. However, viability percentages declined thereafter.

In another study (20), S. platensis biomass showed no influence either on fermentation activity or on growth of B. bifidum or B. animalis when the milk was inoculated with a mixed culture of S. thermophiles and B. bifidum or B. animalis. The data on the viable cell counts of L. bulgaricus showed generally some fluctuations, and in general results, though positive on viability, were somewhat contradictory (21).

In the end, due to the only partially positive and contradictory results, the usefulness of adding whole Spirulina or Chlorella to promote the growth and viability of probiotic strains has been shown to be doubtful, especially also considering that “ . . . this addition adversely affects the sensory attributes of the final product.” Not much more research has been done on the effect of microalgae as symbiotic agents, and “ . . . the effects of the addition of microalgae on in vivo viability of probiotics (instead of in-product evaluations) as well as on probiotics activity (not only assessing the viable bacterial count) is an important and scarcely investigated topic that could be taken into consideration” (22).

### DESCRIPTION OF THE INVENTION

The invention rests on the discovery that adding the specific cyanobacterial pigment phycocyanins, either alone as purified or concentrated from any cyanobacterial microalgae (whatever the cyanobacterial source, phycocyanins are quite similar), or together with the whole Aphanizomenon flos aquae microalgae, to the culture medium in which probiotic strains are grown, increases both their ability to grown and their viability, as well as their health-enhancing properties.

The cyanophyta Aphanizomenon flos-aquae (AFA) is a fresh water unicellular blue-green alga that is consumed as a nutrient-dense food source and for its health-enhancing properties (23; 24; 25). Aphanizomenon flos-aquae (AFA), such as that harvested in Upper Klamath Lake (OR, USA) contains a vast array of nutritional substances (vitamins, minerals, aminoacids, EFA) that can promote a better nutrition also for living probiotic bacteria. Moreover, AFA contains also specific molecules, such as a specific type of phycocyanins characterized also by the presence of phycoerythrocyanin (26), which like all phycocyanins, is endowed with significant anti-oxidant (27) and anti-inflammatory (28) properties; as well as other anti-oxidant molecules such as mycosporine-like amino acids (MAAs), present in all algae. These anti-oxidant properties can contribute to protect the bacteria, during the growth, storing and gastro-intestinal passage phases.

Moreover, AFA has a great advantage relative to other microalgae: its pH is very neutral (about 6.4), and its general flavor and taste is also very neutral, making it a better candidate as an addition to probiotic beverages, yogurts and other food items.

Preparation of AFA Extract and Purified AFA-PCs

To test for the ability of whole AFA algae, an AFA extract and its phycocyanins to promote both the growth and the health-enhancing properties of probiotic bacteria, we have set a series of specific experiments. We started from the whole Aphanizomenon flos-aquae (AFA microalgae) in its dry powder form; and we then proceeded to produce two different substances:
a) a blue extract, produced by suspending the whole AFA in water, letting it seat in the water for 12 hours, thus allowing the separation of a blue liquid from a green-brownish deposit; then collecting the blue supernatant through a pipette; then proceeding to store the liquid in refrigerated conditions at 2°-4° C. The spectrophotometric analysis of the blue supernatant revealed the characteristic peaks of PC at 620 nm (24). This extract has a concentration of approximately 25% phycocyanins;
b) the purified AFA-phycocyanins, produced by the standard purification method: we took a portion of the previously produced blue extract, we first dissolved it in phosphate saline buffer (PBS) pH 7.4 (concentration 10 mg/ml) and centrifuged at 2500×g at 4° C. for 10 min to remove any insoluble material. Subsequently, the extract thus obtained was dried, and then the blue-PC was purified by a single-step chromatographic run using a hydroxyapatite column (Bio-RadLaboratories, CA, USA) as previously described (24; 25). The pure AFA-PC (ratio A620/A280 of 4.78) was finally stored at −20° C.

For this specific prebiotic test, the whole AFA was re-suspended in sterile distillate water and filtered using different pore size filters, first 200 μm and subsequently 0.45 and 0.22 μm (Millipore, Milan, Italy) with a final concentration of 6% (w/v) used in all the experiments. Similarly, the gross Blue Extract and the pure AFA-PC were re-suspended in sterile distillate water, filtered with 0.22 μm membrane pore size (Millipore) and utilized at final concentration of 2% (w/v) in all the experiments.

Bacterial Strains and Culture Conditions

In total, four different lactic acid bacteria (LAB) were used: L. acidophilus DDS-1 (29, 30), which was the strain tested most widely; L. rhamnosus ATCC 53103, B. bifidum ATCC 29521 and L. acidophilus ATCC 4356. The strains were routinely grown in Man Rogosa and Shape agar (MRS) (Oxoid, Milan, Italy) at 37° C. for 24-48 h under microaerophilic conditions (5% O2; 10% CO2; 85% N2).

Three human reference pathogens were used in this study: E. coli O157:H7 ATCC 35150; C. albicans ATCC 14053, and S. aureus ATCC 43387. The strains were routinely maintained in Tryptic Soy Agar (TSA, Oxoid) and Sabouraud dextrose agar (Liofilchem, Roseto degli Abruzzi, Italy) respectively at 37° C. Stock cultures of each strain were keep at −80° C. in Nutrient broth (Oxoid) with 15% of glycerol.

Growth Ability of LAB Strains in Liquid Medium Supplemented with AFA or its Extracts.

The effects of AFA or its extracts on the growth ability of L. acidophilus DDS-1, L. rhamnosus ATCC 53103, B. bifidum ATCC 29521 and L. Acidophilus ATCC 4356 were determined. For this, an overnight exponential culture of each microorganisms (10⁶ cfu/mL) was incubated each into 3 different 200 ml aliquots of MRS broth (Oxoid) with whole AFA, Blue Extract or Pure-PCs under microaerophilic conditions for 24 h a 37° C. MRS broth inoculated with L. acidophilus DDS-1, L. rhamnosus ATCC 53103, B. bifidum ATCC 29521 and L. acidophilus ATCC 4356 were included as a control. At established time points (0, 3, 6, 9, 14, 24, 30, 48, 54, 57, 72 h), aliquots were aseptically removed from each MRS broth culture, diluted in physiological saline solution and plated on MRS agar (Oxoid). After incubation at 37° C. for 24 h under microaerophilic conditions, plates were observed for the enumeration of colony forming unit (cfu/mL). All data were expressed as the mean of three independent experiments performed in duplicate.

L. acidophilus DDS-1 (LA-DDS1) Resistance to Artificial Gastrointestinal Conditions in Liquid Medium Supplemented with AFA or its Extracts.

The acid resistance of LA-DDS1 was examined in MRS broth (Oxoid), adjusted with hydrochloric acid (HCl) to a final pH of 2.5. Briefly, the strain was propagated in MRS broth under microaerophilic conditions for 24 h at 37° C., harvested by centrifugation (3500 rpm for 10 min) and washed twice in phosphate-buffered saline (PBS), pH 7.2. Then, the bacterial suspension was inoculated (10%) into 3 different aliquots of acidified MRS broth with whole AFA, Blue Extract or Pure-PCs, and incubated at 37° C. for 1, 2 and 3 h. L. Acidophilus DDS-1, in acidified MRS broth, was included as control. At each time point, aliquots were aseptically removed, diluted in physiological saline solution, plated on MRS agar (Oxoid) and incubated for 24 h at 37° C. under microaerophilic conditions for the subsequent plate count enumeration (cfu/mL).

The same procedure was performed to test bile tolerance, inoculating the LA-DDS1 strain into MRS broth containing 0.3% (w/v) bile salts (Difco, Becton Drive, USA) with whole AFA, the Blue Extract or Pure-PCs at the above mentioned concentrations. L. acidophilus DDS-1, in MRS broth with 0.3% (w/v) bile salts, was included as control. The incubation was carried out for 1, 2 and 3 h at 37° C. Number of viable bacterial cells was enumerated as describes above.

All data are expressed as mean of three independent experiments performed in duplicate.

Preparation of Cell-Free Culture Supernatants of L. acidophilus DDS-1 (LA-DDS1) in Liquid Medium Supplemented with Whole AFA or its Blue Extract or the Pure-PCs; and of L. rhamnosus ATCC 53103 and B. bifidum ATCC 29521 in Liquid Medium Supplemented with Pure-PC.

L. acidophilus DDS-1 was grown in 200 ml of MRS broth (Oxoid) or MRS broth with whole AFA, the Blue Extract or the pure AFA-PCs at 37° C. for 18 h under microaerophilic conditions. The same procedure was followed for the other three strains, but they were grown in a MRS broth containing only pure AFA-PCs.

At established time points of the bacterial growth (from 24 h to 120 h), cells from each MRS broth culture, were pelleted at 17000 rpm for 15 min at 4° C., adjusted to pH 6.5 with 10 N NaOH and filtered (0.22 μm pore size) to remove any remaining bacteria. The cell-free culture supernatants (CFCSs) were collected and signed as follows: AFA-CFCS (from L. acidophilus DDS-1 grown in MRS broth with AFA at 6%); Blue-CFCS (from L. acidophilus DDS-1 grown in MRS broth with the Blue Extract at 2%). As to the Pure-PCs-CFCS, we had four different sets, each of the four strains, L. acidophilus DDS-1 (LA-DDS1), L. rhamnosus ATCC 53103 (LR), B. bifidum ATCC 29521 (BB) and L. acidophilus ATCC 4356 (LA), grown in MRS broth with Pure-PCs at 2%; CFCS extracted from each of the four LAB grown in MRS broth was also included, and labeled as LA-DDS1-CFCS, LR-CFCS, BB-CFCS and LA-CFCS The aliquots of each CFCS were then kept at −20° C. until use.

Antimicrobial Susceptibility of L. Acidophilus DDS-1's Various CFCSs by Agar Well Diffusion Method.

The antimicrobial activities of LA-DDS1+AFA-CFCS, LA-DDS1+Blue-CFCS and LA-DDS1-AFA-PCs-CFCS were tested using agar well diffusion method (AWDM) according to a known method (31), with several modifications. Briefly, several colonies were drawn from each plate of E. coli O157:H7 ATCC 35150 and C. albicans ATCC 14053, added to 30 mL of TSB (Oxoid) and incubated at 37° C. for 24 h. At this point, 500 μL of each pathogen culture (10⁷ cfu/mL) was added to 20 mL of Nutrient agar (Oxoid) maintained at 50° C., poured into petri dishes, and allowed to solidify for 20 min. Wells of 6 mm in diameter were made on the agar with sterile stainless steel cylinders and 50 μL of each CFCS were dropped into the holes; whole AFA, Blue Extract and pure AFA-PCs solutions were also dropped (50 μL each) in several holes to exclude their antimicrobial activity; LA-DDS1-CFCS was also included as control. After 24 h incubation at 37° C., the diameter of the inhibition zone around each hole was measured and the antimicrobial activity was expressed as the mean of inhibition diameters produced by each CFCS. All the experiments were performed in duplicate.

Antimicrobial Activity of LA-DDS1's Different CFCSs by Killing Studies.

The antimicrobial activity of the different LA-DDS1-CFCSs towards E. coli O157:H7 ATCC 35150 and C. albicans ATCC 14053 was examined by time killing studies. In each of the two experiments, the LA-DDS1-CFCS, extracted from the specific LA-DDS1 grown in MRS broth, was included as control. An exponential culture of each bacterial pathogen (10⁸ cfu/mL, 500 μL) was incubated with or without 500 μL of LA-DDS1-AFA-CFCS, LA-DDS1-Blue-CFCS and LA-DDS1-Pure-AFA-PCs-CFCS at 37° C. After 2, 4 and 8 h of incubation, aliquots were aseptically removed, diluted in physiological saline solution, plated on TSA (Oxoid) and Sabouraud dextrose agar (Liofilchem) and incubated at 37° C. for 24 h. After the incubation period, plates were observed and the colony forming units per milliliter (cfu/mL) for each pathogen were counted.

All data are expressed as mean of three independent experiments. The tests were performed in duplicate.

Antimicrobial Activity of the 3 Other LAB's AFA-PC-CFCS Using Time-Kill Studies

The antimicrobial activity of: LR-AFA-PC-CFCS, BB-AFA-PC-CFCS and LA-AFA-PC-CFCS, this time on three different pathogenic strains, that is E. coli O157:H7 ATCC 35150, S. aureus ATCC 43387 and C. albicans ATCC 14053, was examined by time-kill studies. Briefly, an exponential culture of each bacterial pathogen (about 108 cfu/ml, 500 μL) was incubated with or without 500 μL of Pure-PC-CFCSs at 37° C. After 2, 4 and 8 h of incubation, aliquots were aseptically removed, diluted in physiological saline solution, plated on TSA (Oxoid) and SDA (Liofilchem) and incubated at 37° C. for 24 h. After the incubation period, the plates were observed and the colony forming units per milliliter (cfu/ml) were counted for each pathogen.

Statistical Analysis

Statistical analysis was performed using Prism version 5.0 (GraphPad Software, Inc., La Jolla, Calif., USA). The assumptions for parametric test were checked prior to carry out the analysis. When the assumptions for parametric test where not considered, Mann-Whitney or Kruskall-Wallis non-parametric tests with Dunn's multiple comparison test were applied. P values of <0.05 were considered statistically significant.

Results

Effect of AFA and its Extracts on Growth Ability of L. acidophilus DDS-1.

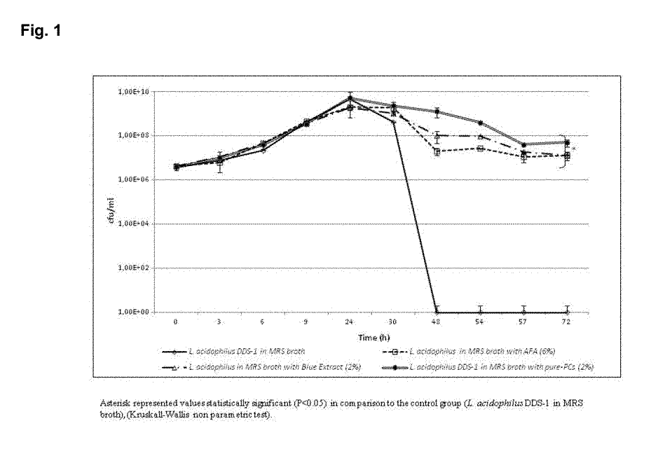

The data relative to the effect of whole AFA (6% w/v), AFA's Blue Extract (2% w/v), and Pure-PCs (2% w/v), on L. acidophilus DDS-1's growth ability are illustrated in FIG. 1. The results showed that culture media containing AFA or its extracts stimulated L. acidophilus DDS-1 growth; in fact, while in MRS broth L. acidophilus DDS-1 stopped growing after 48 h of incubation, in supplemented MRS broths its growth ability was prolonged up to 72 h. Interestingly, the higher growth stimulation was observed in MRS broth containing 2% of Pure-PCs, with cfu/mL values of 5×10⁷ after 72 h.

Effect of AFA and its Extracts on the Growth Ability of the 3 Other LABs.

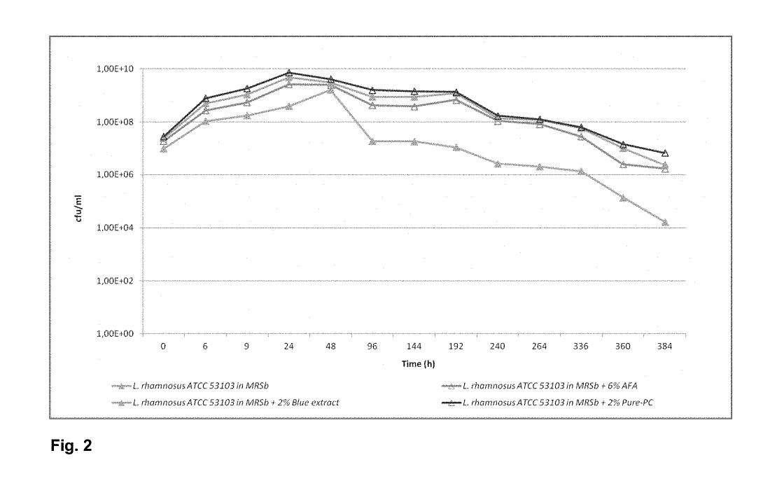

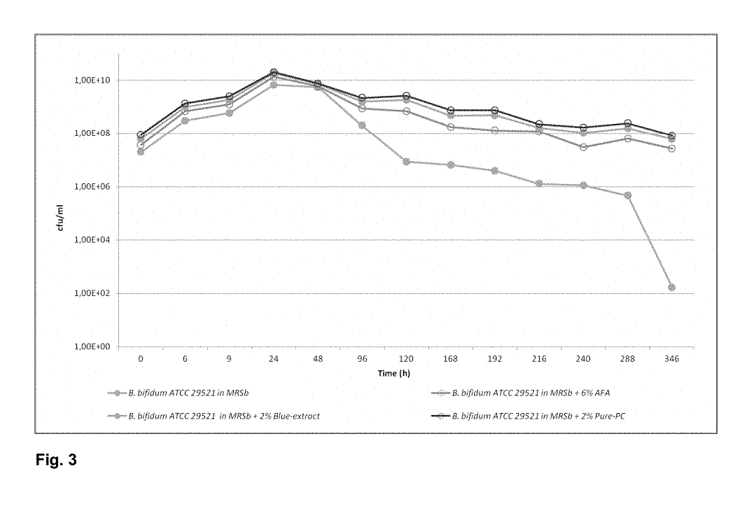

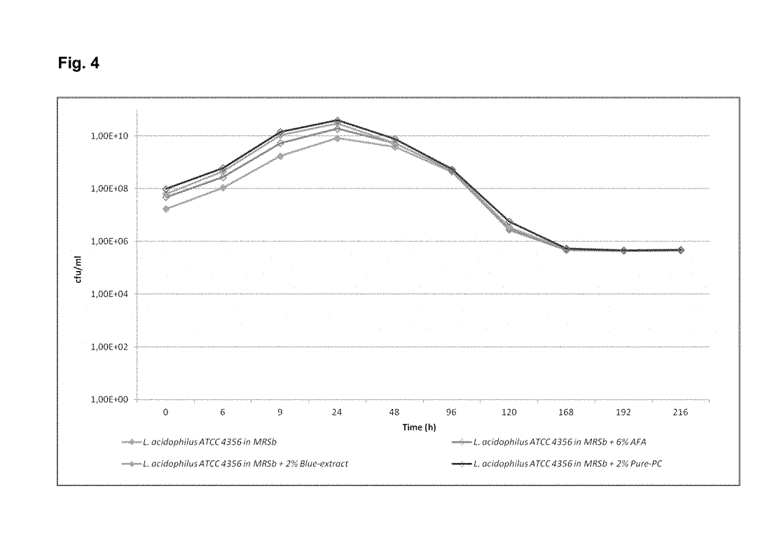

The data regarding the effect of AFA (6% w/v), Blue Extract (2% w/v), and Pure-PCs (2% w/v) on the growth abilities of the different LABs are shown in FIGS. 2-3-4: on L. rhamnosus ATCC 53103 in FIG. 2; on B. bifidum ATCC 29521 in FIG. 3; on L. acidophilus ATCC 4356 in FIG. 4. Culture media containing AFA or its extracts were shown to stimulate particularly the growth of L. rhamnosus ATCC 5310 and B. bifidum ATCC 29521. Indeed, while L. rhamnosus ATCC 53103 decreased growing after 96 h of incubation in MRS broth, its growth ability was prolonged up to 384 h in supplemented MRS broths, with the highest growth stimulation observed in MRS broth containing 2% Pure-PC (4.3×106 cfu/ml). Similarly, the growth of B. bifidum ATCC 29521 was prolonged up to 346 h in MRS broth supplemented with AFA or its extracts, while decreased growing at 120 h in standard MRS broth. A lesser effect was observed in the case of L. acidophilus ATCC 4356, with cfu/ml values quite similar in MRS broth containing AFA or its extracts and MRS broth, during all the examined growth period.

Effect of AFA and its Extracts on Acid and Bile Tolerance of LA-DDS1.

Probiotic must have resistance to low pH and bile salts, in order to survive into the stomach and to perform their health promoting benefit. Here we have tested only the effect of whole AFA, its Blue-Extract and the Pure-PCs at the mentioned concentrations on bile and acid tolerance of L. acidophilus DDS-1. Data are illustrated in Table 1 below:

| | Mean cfu/mL (±sd) after | | |
|---|---|---|---|
| Culture media | 1 h | 2 h | 3 h |
| MRS broth pH 2.5 | 5.01 × 10⁸ (±1.74 × 10⁸) | 5.04 × 10⁸ (±2.0 × 10⁸) | 2.19 × 10⁸ (±2.29 × 10⁷) |
| MRS broth pH 2.5 + 6% AFA | 7.08 × 10⁸ (±1.15 × 10⁸) | 5.37 × 10⁸ (±1.32 × 10⁸) | 3.09 × 10⁸ (±3.47 × 10⁷) |
| MRS broth pH 2.5 + 2% Blue Extract | 5.25 × 10⁸ (±5.75 × 10⁷) | 6.46 × 10⁸ (±3.02 × 10⁷) | 6.61 × 10⁸ (±2.88 × 10⁷) |
| MRS broth pH 2.5 + 2% Pure-PCs | 4.47 × 10⁸ (±1.95 × 10⁸) | 4.37 × 10⁸ (±1.32 × 10⁸) | 2.09 × 10⁸ (±2.40 × 10⁷) |
| MRS broth 0.3% bile salt | 3.24 × 10⁸ (±1.66 × 10⁸) | 2.69 × 10⁸ (±1.82 × 10⁸) | 1.17 × 10⁸ (±5.62 × 10⁷) |
| MRS broth 0.3% bile salts + 6% AFA | 5.62 × 10⁸ (±5.75 × 10⁷) | 7.24 × 10⁸ (±3.72 × 10⁷) | 8.32 × 10⁸ (±1.10 × 10⁸) |
| MRS broth 0.3% bile salts + 2% Blue | 4.57 × 10⁸ (±5.74 × 10⁷) | 2.95 × 10⁸ (±3.89 × 10⁷) | 2.29 × 10⁸ (±1.82 × 10⁷) |
| Extract | | | |
| MRS broth 0.3% bile salts + 2% Pure-PCs | 3.31 × 10⁸ (±1.74 × 10⁸) | 2.51 × 10⁸ (±4.47 × 10⁷) | 1.15 × 10⁸ (±3.89 × 10⁷) |

### LA1 in Acid and Biliary Medium

As reported, L. acidophilus DDS-1 was resistant to low pH and 0.3% bile salts up to 3 h with values of 2.19×10⁸ and 1.17×10⁸ cfu/mL respectively. The presence of AFA or its extracts in the culture media did have some effect on the survival of L. acidophilus DDS-1 to simulated gastric conditions. While the effect wasn't dramatic, it was relatively significant. In relation to acid condition (pH 2.5), while remaining in the same exponential order of 10⁸, while the LA-DDS1 by itself, after 3 hours, descended from 5.01×10⁸ to 2.19×10⁸ cfu/ml, grown with 2% Pure-PCs it actually increased, after 3 hours, to 6.61×10⁸ cfu/ml. In relation to bile salts, it is actually the whole AFA algae to perform best: while LA-DDS1 by itself decreased from 3.24×10⁸ to 1.17×10⁸, when grown with 6% AFA-algae, it actually increased to 8.32×10⁸ cfu/ml.

Antimicrobial Activity of L. acidophilus DDS-1 CFCSs

The antimicrobial activity of AFA-CFCS, Blue-CFCS and Pure-PC-CFCS were tested against E. coli O157:H7 ATCC 35150 and C. albicans ATCC 14053 by AWDM and killing studies using, in the last case, two different experimental designs. As regard AWDM, a remarkable zones of inhibition were observed for all the tested CFCSs against E. coli O157:H7 ATCC 35150 and C. albicans ATCC 14053. However, the presence of AFA or its extracts in the culture media of L. acidophilus DDS-1 enhanced the antimicrobial effect of the relative extracted CFCSs. No inhibition zones were observed around the holes filled with AFA, Blue Extract or Pure-AFA-PCs solutions respectively, as visible in Table 2 below:

| | Zone of inhibition | |
|---|---|---|
| | E. coli O157:H7 | C. albicans |
| CFCSs | ATCC 35150 | ATCC 14053 |
| AFA-CFCS (MRS Broth with | ++ | +++ |
| 6% AFA) | | |
| AFAMAX-CFCS (MRS Broth with | ++ | ++ |
| 2% Blue Extract) | | |
| Blue-PC-CFCS (MRS Broth with | ++ | ++ |
| 2% Pure-PCs) | | |
| LA-CFCS (MRS Broth) | + | + |
| AFA (6%) | | − |
| Blue Extract (2%) | − | − |
| Pure-PCs (2%) | − | − |

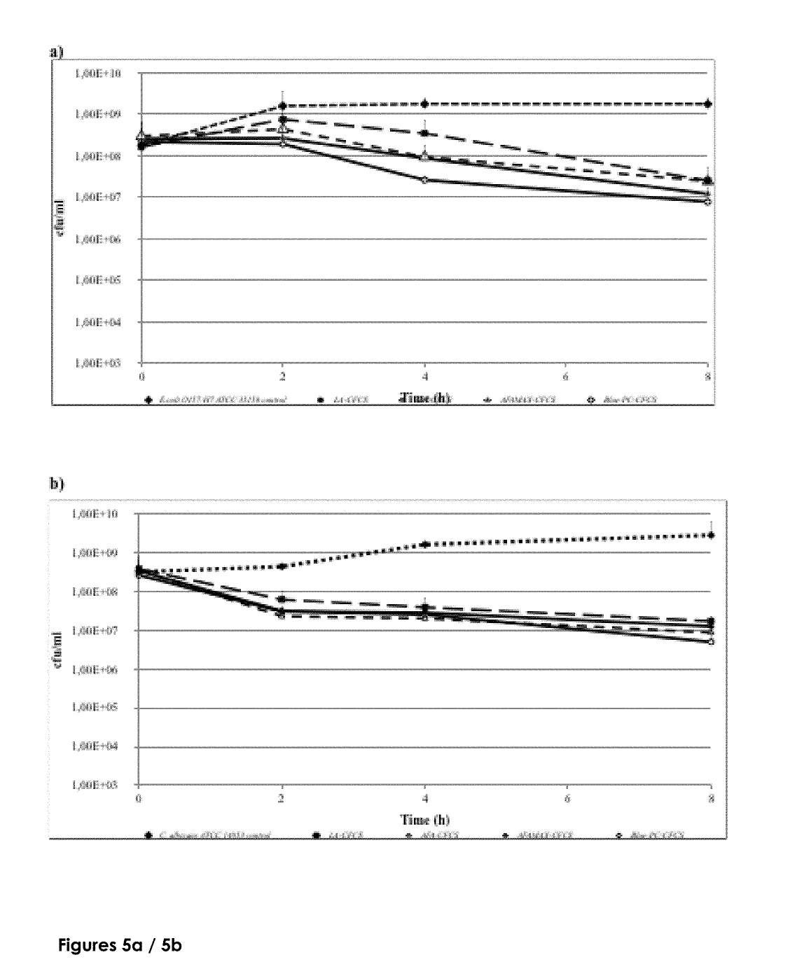

Results of the first experimental test of killing studies were illustrated in FIG. 5 (a-b). Data showed that the presence of AFA, Blue Extract or Pure-PCs in the culture media has maximized the antimicrobial efficacy of L. acidophilus DDS-1 CFCSs against E. coli 0157: H7 ATCC 35150 (FIG. 5a) as demonstrated by the reported cfu/mL values, lesser than to those obtained with the control LA-CFCS. In particular, after 8 h of incubation with Pure-PCs-CFCS, cfu/mL reached values of 7.70×10⁶ in comparison to 2.50×10⁷ cfu/mL obtained with LA-CFCS. Analogously (FIG. 5b), when C. albicans ATCC 14053 was incubated with AFA-CFCS, Blue-CFCS and Pure-PCs-CFCS a positive effect on antimicrobial properties of L. acidophilus DDS-1 was observed. In detail, after 8 h of incubation with Pure-PCs-CFCS a decrease up to 4.97×10⁶ cfu/mL was evidenced, in comparison to 1.70×10⁷ cfu/mL obtained with LA-CFCS.

Results of the second experimental design of killing studies, expressed as logarithmic reduction of pathogenic growth, were illustrated in Tables 3a-b below:

TABLE 3a

| | 2 h | | 4 h | | 8 h | |
|---|---|---|---|---|---|---|
| Time | LA1- | Pure-PCs- | LA1- | Pure-PCs- | LA1- | Pure-PCs- |
| points: | CFCS | CFCS | CFCS | CFCS | CFCS | CFCS |
| 0 h | 0.01 | 0.02 | 0.05 | 0.07 | 0.05 | 0.07 |
| 4 h | 0.80 | 0.85 | 0.64 | 0.82 | 0.80 | 0.89 |
| 6 h | 0.85 | 0.89 | 0.69 | 0.86 | 0.84 | 0.93 |
| 8 h | 1.75 | 1.78 | 0.76 | 1.40 | 0.92 | 1.06 |
| 12 h | 1.58 | 1.59 | 1.12 | 1.89 | 1.80 | 2.03 |
| 24 h | 1.69 | 2.56 | 1.73 | 3.80 | 4.16 | 4.43 |
| 28 h | 1.62 | 2.55 | 2.23 | 3.84 | 3.13 | 3.73 |
| 30 h | 1.64 | 2.34 | 2.19 | 3.89 | 2.23 | 3.46 |
| 48 h | 1.64 | 2.25 | 2.15 | 3.32 | 2.14 | 3.36 |
| 54 h | 1.69 | 1.87 | 2.19 | 3.30 | 2.07 | 3.38 |
| 57 h | 1.75 | 1.76 | 1.77 | 2.96 | 1.39 | 3.65 |
| 72 h | 1.25 | 1.75 | 1.29 | 2.41 | 1.15 | 2.57 |

TABLE 3b

| | 2 h | | 4 h | | 8 h | |
|---|---|---|---|---|---|---|
| Time | LA- | Pure-PCs- | LA- | Pure-PCs- | LA- | Pure-PCs- |
| points: | CFCS | CFCS | CFCS | CFCS | CFCS | CFCS |
| 0 h | 0.03 | 0.04 | 0.04 | 0.04 | 0.06 | 0.07 |
| 4 h | 0.48 | 0.74 | 0.52 | 0.66 | 0.53 | 0.71 |
| 6 h | 0.49 | 0.52 | 0.53 | 0.74 | 0.53 | 0.72 |
| 8 h | 0.70 | 1.68 | 1.25 | 1.99 | 1.23 | 1.71 |
| 12 h | 1.84 | 1.96 | 1.74 | 1.96 | 1.84 | 1.93 |
| 24 h | 1.60 | 3.74 | 1.84 | 3.19 | 1.94 | 3.59 |
| 28 h | 1.73 | 3.67 | 1.31 | 3.57 | 2.09 | 3.78 |
| 30 h | 1.71 | 3.26 | 1.57 | 3.74 | 2.29 | 3.67 |
| 48 h | 1.55 | 3.16 | 1.36 | 3.34 | 1.59 | 3.23 |
| 54 h | 1.77 | 2.87 | 1.30 | 2.72 | 2.15 | 2.20 |
| 57 h | 1.62 | 2.60 | 1.36 | 2.32 | 1.50 | 2.29 |
| 72 h | 1.24 | 2.20 | 1.28 | 2.13 | 1.43 | 2.10 |

In this case, since in the first experiment Pure-PCs showed the most interesting results in stimulating growth and antimicrobial activity of L. acidophilus DDS-1, a total of 24 CFCSs (12 extracted from L. acidophilus DDS-1 grown in MRS broth and 12 in MRS broth with Pure-PCs) extracted at different time points (0, 4, 6, 8, 12, 24, 28, 30, 48, 54, 57, 72 h), were examined by killing studies after 2, 4 and 8 h of incubation against E. coli 0157: H7 ATCC 35150 and C. albicans ATCC 14053.

As regards E. coli 0157: H7 ATCC 35150 (Table 3a), after 2 h of killing incubation, logarithmic reduction of 2.56 was observed with Pure-AFA-PCs-CFCS extracted at time point 24 h, while a less reduction (1.69) was evidenced using the correspondent control LA-CFCS. Similarly, after 4 and 8 h of incubation with Pure-AFA-PCs-CFCS extracted at time point 24 h, increasing logarithmic reduction of 3.8 and 4.43 respectively were evidenced. The antimicrobial activity of Pure-AFA-PCs-CFCSs extracted in the time points from 26 to 57 h was still evident, with logarithmic reductions higher than the corresponding LA-CFCSs. In particular, logarithmic reduction of 2.96 was observed after 4 h of incubation with Pure-AFA-PCs-CFCS extracted at time point 57 h compared to 1.77 log reduction of the correspondent LA-CFCS. Similarly, after 8 h of killing, a logarithmic reduction of 3.65 was obtained using Pure-AFA-PCs-CFCS extracted at time point 57 h in comparison to 1.39 observed with the correspondent LA-CFCS.

As regards C. albicans ATCC 14053, relative data are summarized in Table 3b. As observed for E. coli 0157: H7 ATCC 35150, after 2 h of killing incubation with Pure-AFA-PCs-CFCS extracted at time point 24 h, a logarithmic reduction of 3.74 was registered, higher than 1.60 log reduction obtained with the correspondent LA-CFCS. Similarly, after 4 and 8 h of incubation with Pure-PCs-CFCS extracted at time point 48 h, growth reductions of 3.34 and 3.23 respectively were observed. The Pure-PCs-CFCSs extracted in the later time points (from 54 to 72 h) have showed logarithmic reductions gradually reduced, remaining, in any case, higher compared to the corresponding LA-CFCSs.

Antimicrobial Activity of the 3 LAB's Pure-PC.CFCS

The antimicrobial activity of the different Pure-PC-CFCSs was tested against E. coli O157:H7 ATCC 35150, S. aureus ATCC 43387 and C. albicans ATCC 14053 by time-kill studies. Results of the second time-kill study experimental design, expressed as logarithmic reduction of pathogen growth, are illustrated in Tables 4 a, b, c (the Pure-PC is indicated as “phyco”).

In this study, the antimicrobial activity of L. rhamnosus ATCC 53103, L. acidophilus ATCC 4356 and B. bifidum ATCC 29521 against the different pathogens was enhanced by Pure-PC-CFCS. In fact, in the experiments performed using CFCSs, extracted at different time points during incubation in MRS broth supplemented with Pure-PC, the antimicrobial properties of L. rhamnosus ATCC 53103 and B. bifidum ATCC 29521 were maximized over time.

An evident example is the effect of Pure-PC-CFCSs against E. coli 0157: H7 ATCC 35150 in Table 4a below (“phyco” stands for “PC”):

| gram-negative | log | log reduction | log | log reduction |
|---|---|---|---|---|
| E. coli O157:H7 | 8.31 | | 8.92 | |
| E. coli O157:H7 + CFCS L. rhamnosus T24 | 6.03 | 2.28 | 5.64 | 3.29 |
| E. coli O157:H7 + CFCS L. rhamnosus T48 | 6.56 | 1.74 | 0.00 | 8.92 |
| E. coli O157:H7 + CFCS L. rhamnosus T120 | 6.00 | 2.31 | 0.00 | 8.92 |
| E. coli O157:H7 + CFCS-Phyco L. rhamnosus T24 | 5.78 | 2.53 | 4.43 | 4.50 |
| E. coli O157:H7 + CFCS-Phyco L. rhamnosus T48 | 5.29 | 3.02 | 0.00 | 8.92 |
| E. coli O157:H7 + CFCS-Phyco L. rhamnosus T120 | 0.00 | 8.31 | 0.00 | 8.92 |
| E. coli + CFCS L. acidophilus T24 | 5.95 | 2.35 | 5.73 | 3.20 |
| E. coli + CFCS L. acidophilus T48 | 4.82 | 3.48 | 0.00 | 8.92 |
| E. coli + CFCS L. acidophilus T120 | 6.43 | 1.88 | 0.00 | 8.92 |
| E. coli + CFCS-Phyco L. acidophilus T24 | 6.33 | 1.98 | 6.56 | 2.36 |
| E. coli + CFCS-Phyco L. acidophilus T48 | 5.52 | 2.78 | 5.60 | 3.32 |
| E. coli + CFCS-Phyco L. acidophilus T120 | 6.00 | 2.31 | 2.82 | 6.10 |
| E. coli + CFCS B. bifidum T24 | 5.87 | 2.44 | 6.04 | 2.88 |
| E. coli + CFCS B. bifidum T48 | 5.00 | 3.31 | 3.00 | 5.92 |
| E. coli + CFCS B. bifidum T120 | 5.56 | 2.74 | 0.00 | 8.92 |
| E. coli + CFCS-Phyco B. bifidum T24 | 5.04 | 3.27 | 5.82 | 3.10 |
| E. coli + CFCS-Phyco B. bifidum T48 | 5.43 | 2.88 | 2.00 | 6.92 |
| E. coli + CFCS-Phyco B. bifidum T120 | 5.12 | 3.18 | 0.00 | 8.92 |

A logarithmic reduction of 2.53 was observed after 4 h of incubation with L. rhamnosus ATCC 53103's Pure-PC-CFCS extracted at the 24 h time point, while a smaller reduction (2.28) was obtained using the correspondent CFCS control. Similarly, after 8 h of incubation with L. rhamnosus ATCC 53103's Pure-PC-CFCS extracted at time point 24 h, increased logarithmic reduction of (4.5 log) was observed. The antimicrobial activity of Pure-PC-CFCSs extracted at the time points 48 and 120 h was still evident, with higher logarithmic reductions than those obtained with the corresponding CFCSs. The same trend was evidenced with Pure-PC-CFCSs of B. bifidum ATCC 29521.

Moreover, the effect of Pure-PC-CFCSs was evident against S. aureus ATCC 43387, as for Table 4b below (“phyco” stands for “PC”):

| gram-positive | log | log reduction | log | log reduction |
|---|---|---|---|---|
| S. aureus control | 7.57 | | 8.82 | |
| S. aureus + CFCS L. rhamnosus T24 | 6.03 | 1.54 | 5.78 | 3.05 |
| S. aureus + CFCS L. rhamnosus T48 | 5.60 | 1.97 | 4.87 | 3.96 |
| S. aureus + CFCS L. rhamnosus T120 | 5.22 | 2.35 | 4.78 | 4.05 |
| S. aureus + CFCS-Phyco L. rhamnosus T24 | 6.52 | 1.05 | 4.82 | 4.00 |
| S. aureus + CFCS-Phyco L. rhamnosus T48 | 5.30 | 2.27 | 4.12 | 4.70 |
| S. aureus + CFCS-Phyco L. rhamnosus T120 | 4.82 | 2.75 | 4.60 | 4.22 |
| S. aureus + CFCS L. acidophilus T24 | 5.95 | 1.62 | 5.30 | 3.52 |
| S. aureus + CFCS L. acidophilus T48 | 5.43 | 2.14 | 4.90 | 3.92 |
| S. aureus + CFCS L. acidophilus T120 | 6.43 | 1.14 | 4.87 | 3.96 |
| S. aureus + CFCS-Phyco L. acidophilus T24 | 5.97 | 1.60 | 5.64 | 3.19 |
| S. aureus + CFCS-Phyco L. acidophilus T48 | 5.88 | 1.68 | 4.43 | 4.40 |
| S. aureus + CFCS-Phyco L. acidophilus T120 | 5.00 | 2.57 | 4.00 | 4.82 |
| S. aureus + CFCS B. bifidum T24 | 5.87 | 1.70 | 5.52 | 3.30 |
| S. aureus + CFCS B. bifidum T48 | 5.87 | 1.70 | 5.22 | 3.60 |
| S. aureus + CFCS B. bifidum T120 | 5.56 | 2.01 | 4.99 | 3.84 |
| S. aureus + CFCS-Phyco B. bifidum T24 | 5.78 | 1.79 | 5.52 | 3.30 |
| S. aureus + CFCS-Phyco B. bifidum T48 | 5.85 | 1.72 | 4.64 | 4.19 |
| S. aureus + CFCS-Phyco B. bifidum T120 | 5.12 | 2.44 | 4.52 | 4.30 |

A logarithmic reduction of 2.27 was observed after 4 h of incubation with L. rhamnosus ATCC 53103's Pure-PC-CFCS extracted at the 48 h time point, in comparison to 1.97 obtained using the correspondent CFCS control. Similarly, after 8 h of incubation with L. rhamnosus ATCC 53103's Pure-PC-CFCS extracted at time point 120 h a logarithmic reduction of 2.75 was observed. The same trend was evidenced with the Pure-PC-CFCSs of B. bifidum ATCC 29521.

Data for C. albicans ATCC 14053 are summarized in Table 4c. below (“phyco” stands for “PC”):

| micete | log | log reduction | log | log reduction |
|---|---|---|---|---|
| C. abicans control | 6.24 | | 7.05 | |
| C. abicans + CFCS L. rhamnosus T24 | 4.82 | 1.41 | 5.64 | 1.42 |
| C. albicans + CFCS L. rhamnosus T48 | 4.12 | 2.11 | 4.08 | 2.98 |
| C. albicans + CFCS L. rhamnosus T120 | 3.52 | 2.72 | 4.00 | 3.05 |
| C. abicans + CFCS-Phyco L. rhamnosus T24 | 3.67 | 2.57 | 3.85 | 3.21 |
| C. abicans + CFCS-Phyco L. rhamnosus T48 | 3.43 | 2.81 | 3.43 | 3.63 |
| C. abicans + CFCS-Phyco L. rhamnosus T120 | 4.12 | 2.11 | 3.82 | 3.23 |
| C. abicans + CFCS L. acidophilus T24 | 4.12 | 2.11 | 5.08 | 1.98 |
| C. abicans + CFCS L. acidophilus T48 | 3.01 | 3.22 | 0.00 | 7.05 |
| C. abicans + CFCS L. acidophilus T120 | 2.52 | 3.71 | 3.12 | 3.93 |
| C. abicans + CFCS-Phyco L. acidophilus T24 | 3.70 | 2.54 | 4.90 | 2.15 |
| C. abicans + CFCS-Phyco L. acidophilus T48 | 3.64 | 2.60 | 3.64 | 3.42 |
| C. abicans + CFCS-Phyco L. acidophilus T120 | 5.56 | 0.67 | 3.22 | 3.83 |
| C. abicans + CFCS B. bifidum T24 | 4.12 | 2.11 | 4.37 | 2.69 |
| C. abicans + CFCS B. bifidum T48 | 3.30 | 2.94 | 3.52 | 3.54 |
| C. abicans + CFCS B. bifidum T120 | 5.56 | 0.67 | 3.28 | 3.78 |
| C. abicans + CFCS-Phyco B. bifidum T24 | 3.37 | 2.87 | 3.00 | 4.05 |
| C. abicans + CFCS-Phyco B. bifidum T48 | 3.22 | 3.02 | 3.22 | 3.83 |
| C. abicans + CFCS-Phyco B. bifidum T120 | 5.00 | 1.24 | 3.22 | 3.83 |

As had been observed for E. coli 0157: H7 ATCC 35150 and S. aureus ATCC 43387, after 4 h of incubation with B. bifidum ATCC 29521 Pure-PC-CFCS extracted at time point 24 h, a logarithmic reduction of 2.87 was recorded, higher than the 2.84 log reduction obtained with the corresponding CFCS. Similarly, after 8 h of incubation with Pure-PC-CFCS extracted at time point 24 h, logarithmic reductions of 4.05 and was observed compared to 2.69 of the relative control.

Discussion

Effect of AFA and its Extracts on Growth Ability of L. acidophilus DDS-1.

As visible from FIG. 1, growing LA-DDS1 together with AFA or its extracts promoted a significantly higher growth. This stands out particularly in comparison with the results obtained by adding Spirulina in the bacterial culture. As reported by De Caire et al. (2000), the two Lactobacillus strains with added Spirulina in the growth medium reached the growth plateau at 10 h, and kept growing for up to 20 h. However, the only difference with the strains without Spirulina was only the speed of growth, because at 20 h the latter reached the same growth level of the Spirulina enriched culture, which shows that the effect of adding Spirulina to the medium was indeed not so useful. On the contrary, adding AFA Klamath algae to the medium generates much deeper results. The DDS-1 strain is in fact stronger than the strains tested by the De Caire et el., as it kept growing for up to more than 30 h. then decreasing very rapidly in the following time reaching zero at 48 hrs. But the Klamath algae enriched DSS-1 strain, maintained a high growth curve for up to 72 h, and even at 72 h there was no sign of decrease. Even better was the result achieved with the Pure AFA's PCs. Similar results have been obtained with the other 3 strains, L. rhamnosus ATCC 53103, L. acidophilus ATCC 4356 and B. bifidum ATCC 29521, as shown in FIGS. 2,3 and 4. Here, the growth kept going even after 346 hours with both L. Rhamnosus and B Bifidum (the effect on L. Acidophilus ATCC 4356 was significantly less marked).

Effect of AFA and its Extracts on Acid and Bile Tolerance of L. acidophilus DDS-1

As shown in Table 1, the addition of AFA or its extracts improves the innate ability of the L. acidophilus DDS1 to survive the pH variations of the gastro-intestinal tract or the presence of bile. The strain is quite resistant on its own to acid and bile conditions, but while the strain by itself in acid conditions decreases its cfu count from 5.01 to 2.19×10⁸, when grown with the Blue phycocyanin extract, it actually increases the cfu count from 5.25 to 6.61×10⁸; and while the strain by itself, when in contact with bile salts, decreases its cfu count from 3.24 to 1.17×10⁸, when grown with the whole AFA algae it actually increases its cfu count from 5.62 to 8.32×10⁸.

Antimicrobial Activity of L. acidophilus DDS-1 CFCSs, as Well as of the Three Other LAB Strains.

This is one of the most essential activities of probiotics, and here both AFA algae and phycocyanins cause a significant increase in the ability of L. acidophilus DDS1 to fight and kill pathogens such as E. coli and C. albicans. In the first testing module, as shown in Table 2, showing the Agar Well Diffusion Method (AWDM), the zones of inhibition for E. coli O157:H7 ATCC 35150 were doubled, relative to control (L. acidophilus DDS1 by itself), by all three substances, whole AFA, the Blue Extract and the Pure AFA-PCs; whereas in relation to C. albicans ATCC 14053 the best result was obtained by adding the whole AFA in the culture. This gives a first indication that, as refers to antimicrobial activity, adding the whole AFA to the probiotic broth may be the best choice. However, in the second testing model performing killing studies, as shown in FIGS. 3 a-b, while all the substances, including the L. acidophilus DDS1 by itself, are able to kill substantial numbers of pathogens, clearly the best performance is generated, both against E. coli and C. albicans, by the mix obtained by adding the Pure-AFA-PCs to the growing broth. The higher ability of the Pure-AFA-PCs to generate a probiotic capable of better killing pathogens is further confirmed by the count of the logarithmic reduction of the pathogens shown on Table 3. Here we can see that as against E. coli O157:H7 ATCC 35150, L. acidophilus DDS1 incubated for 24 h with the Pure AFA-PCs manages to reach a very significant reduction, much higher that the control (L. acidophilus DDS1), already after 4 h of contact with the pathogen; while the most significant increase is seen with C. albicans, where the best result again is obtained by the broth extracted after 24 h of incubation with Pure-AFA-PCs, and only after 2 h of contact with the pathogen.

As to the three other strains, given the LA-DDS1's CFCSs performance with the Pure-PC was the best, we tested them also with the addition to the culture of Pure-PC to obtain that Pure-PC-CFCS. The results confirmed the ability of Pure-PCs to significantly increase the LAB's killing ability in relation to E. Coli, S. Aureus and C. Albicans, as shown in Tables 4 a, b, c.

### REFERENCES

1) Food and Agriculture Organization of United Nations and World Health Organization Working Group (FAO/WHO) (2002). Guidelines for the Evaluation of Probiotics in Food; Technical report for FAO/WHO: London, UK.
2) Bergonzelli, G. E., Blum, S., Brussow, H., & Corthesy-Theulaz, I. (2005). Probiotics as a treatment strategy for gastrointestinal diseases? Digestion 72, 57-68.
3) Ventura, M., O'Flaherty, S., Claesson, M. J., Turroni, F., Klaenhammer, T. R., van Sinderen, D., & O'Toole, P. W. (2009). Genome-scale analyses of health-promoting bacteria: pro-biogenomics. Nature Reviews. Microbiology 7, 61-71.
4) Likotrafiti, E., Manderson, K. S., Fava, F., Tuohy, K. M., Gibson, G. R., & Rastall, R. A. (2004). Molecular identification and anti-pathogenic activities of putative probiotic bacteria isolated from faeces of healthy elderly individuals. Microbial Ecology in Health and Disease 16, 105-112.
5) Teitelbaum, J. E., & Walker, W. A. (2002). Nutritional impact of pre- and probiotics as protective gastrointestinal organisms. Annual Review of Nutrition 22, 107-138.
6) Lupton, J. (2004). Microbial degradation products influence colon cancer risk: The butyrate controversy. Journal of Nutrition 134, 479-482.
7) Kunová, G., Rada, V., Lisová, I., Ročková, Š., & Vilková, E. (2011). In vitro fermentability of prebiotic oligosaccharides by Lactobacilli. Czech Journal of Food Science 29 (Special Issue), 49-54.
8) Patel, S., & Goyal, A. (2012). The current trends and future perspectives of prebiotics research: a review. 3 Biotech 2, 115-125.
9) Blaiotta, G., La Gatta, B., Di Capua, M., Di Luccia, A., Coppola, R., & Aponte, M. (2013). Effect of chestnut extract and chestnut fiber on viability of potential probiotic Lactobacillus strains under gastrointestinal tract conditions. Food Microbiology 36, 161-169.
10) Macfarlane, G. T., Steed, H., & Macfarlane, S. (2008). Bacterial metabolism and health-related effects of galacto-oligosaccharides and other prebiotics. Journal of Applied Microbiology 104, 305-344.
11) Fujimori, S., Gudis, K., Mitsui, K., Seo, T., Yonezawa, M., Tanaka, S., Tatsuguchi, A., & Sakamoto, C. (2009). A randomized controlled trial on the efficacy of synbiotic versus probiotic or prebiotic treatment to improve the quality of life in patients with ulcerative colitis. Nutrition 25, 520-525.
12) Liong, M. T. (2008). Roles of probiotics and prebiotics in colon cancer prevention: postulated mechanisms and in vivo evidence. International Journal of Molecular Sciences 9, 854-863.
13) Saulnier, D. M., Gibson, G. R., & Kolida, S. (2008). In vitro effects of selected synbiotics on the human faecal microbiota composition. FEMS Microbiology Ecolology 66, 516-27.
14) Grimoud, J., Durand, H., Courtin, C., Monsan, P., Ouarné, F., Theodorou, V., & Roques, C. (2010). In vitro screening of probiotic lactic acid bacteria and prebiotic glucooligosaccharides to select effective synbiotics. Anaerobe 16, 493-500.
15) Adamberg, S., Sumeri, I., Uusna, R., Ambalam, P., Kondepudi, K. K., Adamberg, K., Wadstro, T., & Ljungh A. (2014). Survival and synergistic growth of mixed cultures of bifidobacteria and lactobacilli combined with prebiotic oligosaccharides in a gastrointestinal tract simulator. Microbial Ecology in Health & Disease.
16) Pranckutė, R., Kaunietis, A., Kuisienė, N., & Čitavičius, N. D. (2014). Development of synbiotics with inulin, palatinose, α-cyclodextrin and probiotic bacteria. Polish Journal of Microbiology 63, 33-41.
17) De Caire, G. Z., Parada, J. L., Zaccaro, M. C., & de Cano, M. M. S. (2000). Effect of Spirulina platensis biomass on the growth of lactic acid bacteria in milk. World Journal of Microbiology and Biotechnology 16, 563-565.
18) Molnár N, Gyenis B, Varga L, (2005). Influence of a powdered Spirulina platensis biomass on acid production of lactococci in milk. Milchwissenschaft 60(4): 380-2.
19) Molnár N, Sipos-Kozma Zs, Tóth A, Asványi B, Varga L, (2009). Development of a functional dairy food enriched with Spirulina (Arthrospira platens). Tejgazdaság 69(2): 15-22.
20) Varga L, Szigeti J, Ördög V. 1999a. Effect of a Spirulina platens biomass enriched with trace elements on combinations of starter culture strains employed in the dairy industry. Milchwissenschaft 54(5):247-8.
21) Beheshtipour, H., Mortazavian A. M., Haratian P., & Darani, K. K. (2012). Effects of Chlorella vulgaris and Arthrospira platensis addition on viability of probiotic bacteria in yogurt and its biochemical properties. European Food Research and Technology 235, 719-728.
22) Beheshtipour et al., Supplementation of Spirulina platensis and Chlorella vulgaris Algae into Probiotic Fermented Milks, in Comprehensive Reviews in Food Science and Food Safety, Mar. 11, 2013.
23) Pugh, N., Ross, S. A., ElSohly, H. N., ElSohly, M. A., & Pasco, D. S. (2001). Isolation of three high molecular weight polysaccharide preparations with potent immunostimulatory activity from Spirulin platensis, Aphanizomenon flos-aquae and Chlorella pyrenoidosa. Planta Medica 67, 737-742.
24) Benedetti, S., Benvenuti, F., Pagliarini, S., Francogli, S., Scoglio, S., & Canestrari, F. (2004). Antioxidant properties of a novel phycocyanin extract from the blue-green alga Aphanizomenon flos-aquae. Life Sciences 75, 2353-2362.
25) Scoglio, S., Benedetti, Y., Benvenuti, F., Battistelli, S., Canestrari, F., Benedetti, S. (2014). Selective monoamine oxidase B inhibition by an Aphanizomenon flos-aquae extract and by its constitutive active principles phycocyanin and mycosporine-like amino acids. Phytomedicine 21, 992-997.
26) Benedetti S., Rinalducci S., Benvenuti F. et al., Purification and characterization of phycocyanin from the bye-green alga Aphanizomenon flos-aquae, Journal of Chromatography B (2006), 833, 12-18.
27) Fernandez-Rojas, B., Medina-Campos, O. N., Hernandez-Pando, R., Negrette-Guzman, M., Huerta-Yepez, S., & Pedraza-Chaverret J. (2014). C-Phycocyanin prevents cisplatin-induced nephrotoxicity through inhibition of oxidative stress. Food & Function 5,480-490.
28) Hwang, J. H., Chen, J. C., Chan Y. C. (2013). Effects of C-phycocyanin and Spirulina on salicylate-induced tinnitus, expression of NMDA receptor and inflammatory genes. PLoS ONE 8(3): e58215. doi:10.1371/journal.pone.0058215.
29) Murthy, M., et al., Delineation of Beneficial Characteristics of Effective Probiotics, in JANA, Vol. 3, No. 2, 2000, pgs. 38-43.
30) Shahani, K. et al., Natural Antibiotic Activity of Lactobacillus Acidophilus and Bulgaricus II, in Cultured Dairy Products Journal, Vol. 12 (2): 8-11, 1977.
31) Campana, R., Federici, S., Ciandrini, E., & Baffone W. (2012). Antagonistic activity of L. acidophilus ATCC 4356 on the growth and on adhesion/invasion characteristics of human C. jejuni. Current Microbiology 64, 371-378.

1. The use of the phycocyanins, preferably from the Aphanizomenon flos-aquae micro-algae, and from any other cyanobacterial blue-greens algae, as an addition to the culture of any probiotic strain, or to any probiotic strain after it has been cultured, working as a pre-biotic to enhance the probiotic strain's viability, growth, resistance to bile and acid conditions, and ability to kill intestinal pathogens, including but not limited to, E. Coli, Staphylococcus aureus and Candida albicans.

2. The use, according to claim 1, whereas the phycocyanins added to the probiotic strains are in their purified form.

3. The use, according to claim 1, whereas the phycocyanins added to the probiotic strain are concentrated from cyanobacterial micro-algae through any concentration and extraction method, such as through water or other organic solvents.

4. The use, according to claim 1, whereas the phycocyanins added to the probiotic strain are naturally contained in the whole cyanobacterial micro-algae, preferably but not limited to the Aphanizomenon flos aquae strain, especially in relation to the ability to enhance the probiotic strain's ability to kill pathogens, and to the ability to better survive the passage through the biliary salts.

5. The use, according to claim 4, whereas the product resulting by adding the phycocyanins and/or the cyanobacterial microalgae to the probiotic strain or strains either during or after the culturing phase, is added in liquid form to any drink, yogurt, or food for its health-enhancing properties; or to any liquid nutraceutical or pharmaceutical product, including vials and creams.

6. The use, according to claim 5, whereas the liquid or cream form is applied topically for skin disorders; or vaginally, or anally, for vaginal or anal bacterial infections.

7. The use, according to claim 6, whereas the product resulting by adding the phycocyanins to the probiotics either before or after culturing them, is dried to obtain a powder which is then added to any food, nutraceutical or pharmaceutical product.

8. The use, according to claim 7, whereas the dried power is manufactured in sachets, tablets or capsules or in any other nutraceutical or pharmaceutical form.

9. The use, according to claim 8, whereas the subjects to whom the probiotic products are administered are animal or human.
